(19) United States
(12) Patent Application Publication
(10) Pub. No.: US 2007/0032696 A1
(43) Pub. Date: Feb. 8, 2007

(54) Cardiac harness delivery device

(76) Inventor: Sieu Duong, Campbell, CA (US)

Correspondence Address: FULWIDER PATTON, 6060 CENTER DRIVE, 10TH FLOOR, LOS ANGELES, CA, 90045, US

(21) Appl. No.: 11/187,607

(22) Filed: Jul. 22, 2005

(51) Int. Cl. A61F 2/00

(52) U.S. Cl. 600/37

(57) ABSTRACT

An apparatus for delivering a cardiac harness onto a heart includes an elongate body and a plurality of elongate push rods longitudinally movable with respect to the elongate body. The elongate body has a tubular housing that is sized to contain the cardiac harness and has a compressible cross-sectional shape. The cardiac harness is releaseably attached to the push rods such that advancement of the push rods in a distal direction moves the cardiac hearness from a compacted configuration in the housing to an expanded configuration outside the housing. The housing may include a plurality of flexible slats that extend longitudinally such that the housing may be compressed to a reduced cross-sectional shape to allow it to advance through a minimally invasive surgical entry path to the heart. The slats may include curved end portions for locking underneath a pericardial sac surrounding the heart.

### FIELD OF THE INVENTION

The present invention relates generally to a device for delivering a cardiac harness onto the heart of a patient.

### BACKGROUND OF THE INVENTION

Congestive heart failure (“CHF”) is characterized by the failure of the heart to pump blood at sufficient flow rates to meet the metabolic demand of tissues, especially the demand for oxygen. It has been determined that a passive wrap, or cardiac harness, may increase the efficiency of a heart affected by congestive heart disease. While advances have been made in cardiac harness technology, a satisfactory device for delivering and positioning the cardiac harness onto a patient's heart has yet to be provided.

In one method, access to a patient's heart is achieved through an open chest procedure, wherein the sternum is split and separated to allow access to the heart. The cardiac harness is then positioned over the heart by manual manipulation. Such an open chest procedure is highly traumatic to the patient and, thus, remains a relatively undesirable option for cardiac harness delivery. Present cardiac harness delivery devices are adapted for use in minimally invasive procedures in which the delivery devices are advanced through a relatively small incision through the body cavity of a patient. Because of the relatively rigid structure and large size of such delivery devices, separate introducer devices are used to create an entry path sufficient in size to allow the delivery device to access the heart. In addition, access to the apex of the heart is typically required, in which case an entry path that passes between two ribs is convenient. Because the space that can be created between two ribs is limited, advancement of present delivery devices between two ribs is often difficult and may strain the ribs excessively.

### SUMMARY OF THE INVENTION

Accordingly, a need exists for a cardiac harness delivery device that that overcomes the disadvantages of the prior art in providing access of a cardiac harness delivery device to the heart. A delivery device that requires no introducer device and has a cross-sectional shape that is collapsible has the advantage of squeezing through narrow passages that may arise in a variety of minimally invasive surgical entry pathways to the heart.

In one aspect of the invention, an apparatus for delivering a cardiac harness onto a heart includes: elongate body with a distal portion having a tubular housing sized to contain the cardiac harness in a compacted configuration, and with an outer surface defining a collapsible cross-sectional shape; and a plurality of elongate push rods longitudinally movable with respect to the elongate body. The cardiac harness is releasably connected to each of the push rods such that advancement of the push rods in a distal direction moves the cardiac harness from the compacted configuration in the housing to an expanded configuration outside the housing.

In another aspect of the invention, the cross-sectional shape of the housing is of a size suitable for a minimally invasive procedure. In further aspects, the housing is substantially cylindrical with a diameter less than 5.1 cm (2 inches). The diameter is less than 3.2 cm (1.25 inches) in an even further aspect of the invention.

In yet another aspect, an inner surface of the housing defines a plurality of channels sized and shaped to receive the push rods. In a further aspect, each of the channels has a surface that defines a cross-section having a first shape and each of the push rods has a surface that defines a cross-section having a second shape that is substantially the same as the first shape. In a detailed aspect, at least one of the first shape and the second shape comprises a dovetail form.

Another aspect of the invention includes housing with a plurality of elongate slats extending longitudinally to the distal end of the housing. In this aspect, the slats are spaced apart, forming a perimeter around the housing. In a further aspect, the slats are biased to form a smaller perimeter at the distal end of the housing relative to the proximal end of the housing. In another aspect, the slats include curved end portions at the distal end of the housing. The curved end portions are configured to temporarily lock the distal end of the housing in an incision in a pericardial sac surrounding the heart. In detailed aspects, the slats have a length greater than 50% of a distance between the proximal end and the distal end of the housing. In more detailed aspects, the length of the slats is greater than 70% of the same distance.

In another aspect, the housing has a substantially circular cross-sectional shape having a diameter. In this aspect, at least a portion of the housing is compressible to a substantially elliptical cross-sectional shape having a minor axis that is less than the diameter. In yet another aspect, the housing has a cross-sectional shape having a first perimeter. At least a portion of the housing is compressible to a reduced cross-sectional shape having a second perimeter that is less than the first perimeter.

An aspect of the invention includes an annular biasing member coupled to the outer surface of the housing. In this aspect, the biasing member compresses a portion of the housing to a smaller cross-sectional shape relative to an uncompressed portion of the housing.

In one aspect of the invention, an apparatus for delivering a cardiac harness onto a heart includes: a tubular housing sized to contain the cardiac harness in a compacted configuration, the housing having an outer surface defining a collapsible cross-sectional shape; and an elongate support member longitudinally movable with respect to the housing, the cardiac harness releasably connected to the support member. In a further aspect, the cross-sectional shape is of a size suitable for a minimally invasive procedure. In yet a further aspect, the support member comprises a plurality of flexible push rods having a sliding, interlocking relationship with the inner surface of the housing.

In another aspect, the housing comprises a plurality of longitudinal slats spaced part in a circumferential arrangement around the housing. In yet another aspect, the slats are biased in a smaller circumferential arrangement at the distal end of the housing relative to the proximal end of the housing.

Another aspect of the invention includes slats that include curved end portions at the distal end of the housing. The curved end portions are sized to advance through an incision in a pericardial sac surrounding the heart and configured to push in a distal direction against a portion of the pericardial sac adjacent to the incision.

In yet another aspect, the housing has a cross-sectional shape having a first dimension. The first dimension is equivalent to the shortest possible linear distance between any two points on the perimeter of the cross-sectional shape and passing through the center of the cross-sectional shape. In this aspect, at least a portion of the housing is collapsible to a reduced cross-sectional shape having a second dimension that is less than the first dimension. The second dimension is equivalent to the shortest possible linear distance between any two points on the perimeter of the reduced cross-sectional shape and passing through the center of the reduced cross-sectional shape.

In another aspect, the housing tapers from a first cross-sectional shape at the proximal end of the housing to a second cross sectional shape at the distal end of the housing. In this aspect, the perimeter of the second cross-sectional shape is smaller than the perimeter of the first cross-sectional shape.

Another aspect of the invention involves an apparatus for delivering a cardiac harness onto a heart that includes a housing. The housing has an outer wall with a flexible portion defining a reduced orientation having a first diameter. The first diameter is of a size sufficient to permit the apparatus to pass through a minimally invasive surgical entry path. In this aspect, the apparatus also includes a support member sized and shaped to be contained in the housing. The cardiac harness releasably connected to the support member of this aspect such that longitudinal movement of the support member in a distal direction (a) moves the cardiac harness from the compacted configuration in the housing to an expanded configuration outside the housing, and (b) urges the flexible portion into an expanded orientation having a second diameter that is larger than the first diameter.

### BRIEF DESCRIPTION OF THE DRAWINGS

These and other features, aspects and advantages of the present invention are described with reference to drawings of a preferred embodiment, which are intended to illustrate, but not to limit, the present invention.

FIG. 1 is a perspective view of a cardiac harness delivery device constructed in accordance with certain features, aspects and advantages of the present invention. The illustrated delivery device comprises a body portion, including an elongate shaft and a housing, and a movable portion, including a control assembly and a plurality of elongate push rods. A cardiac harness is carried by distal end portions of the plurality of push rods.

FIG. 2 is an enlarged, partial cutaway view of a distal portion of the delivery device of FIG. 1 showing the cardiac harness in a compacted configuration within a cavity defined by the housing.

FIG. 3 is a perspective view of the delivery device of FIG. 1 with the movable portion in an advanced position relative to the body portion.

FIG. 4 is an enlarged view of a distal portion of the delivery device of FIG. 1 indicated by line 4-4 of FIG. 3.

FIG. 5 is a side elevational view of the delivery device of FIGS. 1-4, with a pump member, or, specifically, a syringe, attached to a suction assembly of the delivery device. The suction assembly includes a suction cup member, which is configured to securely hold the heart relative to the delivery device during advancement of the cardiac harness over the heart.

FIG. 6 is a side elevational view of the delivery device of FIG. 5 with the cardiac harness in a partially advanced position.

FIG. 7 is a side elevational view of the delivery device of FIG. 5 with the cardiac harness in a fully advanced position and the releasing member being actuated to release the cardiac harness from the delivery device.

FIG. 8 is a side elevational view of the delivery device of FIG. 5 with the cardiac harness being completely released and the plurality of push rods being retracted.

FIG. 9 is a side elevational view of the delivery device of FIG. 5 with the cardiac harness completely released and illustrating the delivery device being withdrawn from the heart.

FIG. 10 is an enlarged side elevational view of the distal portion of a delivery device showing the housing having a plurality of flexible slats.

FIG. 11 is a plan view of the housing of FIG. 10 taken in the direction of line 11-11 of FIG. 10 showing a plurality of channels for receiving the push rods.

FIG. 12 is a cross-sectional view of the housing of FIG. 12 taken in the direction of line 12-12 of FIG. 10 showing an uncompressed cross-sectional shape formed by a circumferential arrangement of the slats.

FIG. 13 is a cross-sectional view of the housing of FIG. 12 taken in the direction of line 12-12 of FIG. 10 showing a compressed cross-sectional shape in response to compressive forces are applied to the slats.

FIG. 14 is a cross-sectional view of the housing of FIG. 12 taken in the direction of line 12-12 of FIG. 10 showing a compressed cross-sectional shape that is substantially elliptical.

FIG. 15 is a perspective, cut-away view of the housing of FIG. 10 taken in the direction of line 15-15 of FIG. 12.

FIG. 16 is an enlarged side elevational view of a distal portion of a delivery device showing a housing with flexible slats being compressed by an annular biasing member.

FIG. 17 is a perspective view of a heart having a small incision in the pericardium to permit the delivery device to access the heart, and sutures having pull strings arranged around the incision.

FIG. 18 is an enlarged side elevational view of a distal portion of the delivery device of FIG. 16 showing a distal end of the housing inserted through the incision and into a space between a portion of a pericardial sac surrounding the heart and the epicardial surface of the heart.

FIG. 19 is an enlarged side elevational view of a distal portion of the delivery device showing a housing with flexible slats having curved end portions.

FIG. 20 is an enlarged side elevational view of a distal portion of the delivery device of FIG. 19 showing the curved end portions inserted through the incision and pressing against the inner surface of a portion of the pericardial sac surrounding the incision.

### DETAILED DESCRIPTION OF THE PREFERRED EMBODIMENTS

The exemplary figures illustrate a preferred embodiment of a cardiac harness delivery device, which is generally referred to by the reference numeral 30. In a preferred embodiment, the delivery device 30 is configured to releasably support a cardiac reinforcement device (CRD), such as a cardiac harness, and assist in the advancement of the cardiac harness over the heart of a patient. Once the cardiac harness is positioned on the heart, the delivery device 30 preferably is configured to release the harness and be retractable without causing undesired shifting of the cardiac harness relative to the heart.

In the illustrated arrangement, the delivery device 30 permits delivery of a cardiac harness in a minimally invasive manner. That is, preferably the device 30 permits accurate delivery, positioning, and release of the cardiac harness through a relatively small incision in a patient. However, the preferred, or alternative, embodiments of the delivery device 30 may also be used to deliver a cardiac harness in an open chest, or other minimally invasive procedure. Further, an embodiment preferably is configured to enable indirect visualization of at least portions of the device 30 during surgery. For example, portions of the device may be radiopaque so as to be visualized and guided by fluoroscopy or other methods.

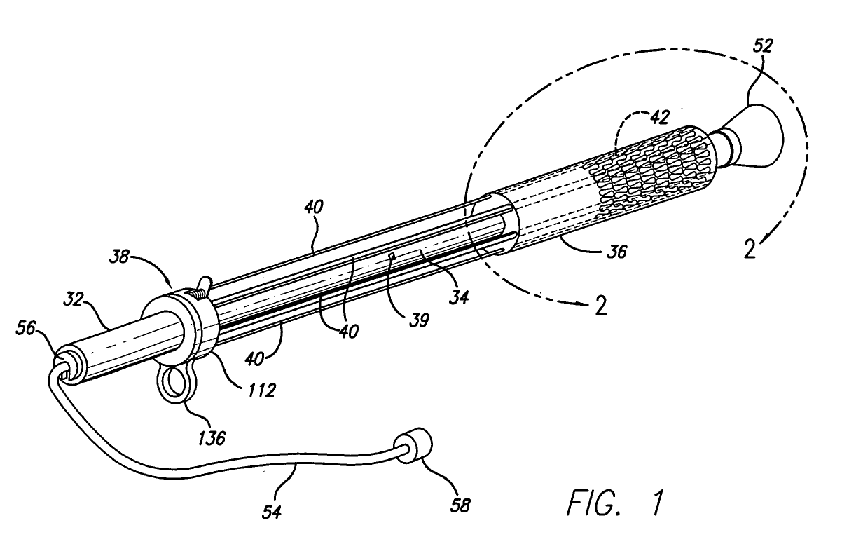

With specific reference to FIG. 1, the illustrated delivery device 30 generally includes a body portion comprised of a handle 32 affixed to the proximal end of a hollow, elongate shaft 34. Preferably, a housing 36 is affixed to a distal end of the elongate shaft 34. The illustrated delivery device 30 also includes a movable portion comprised of a control assembly 38 and a plurality of elongate push rods 40. The control assembly 38 and, thus, the push rods 40, are axially slidable along the shaft 34.

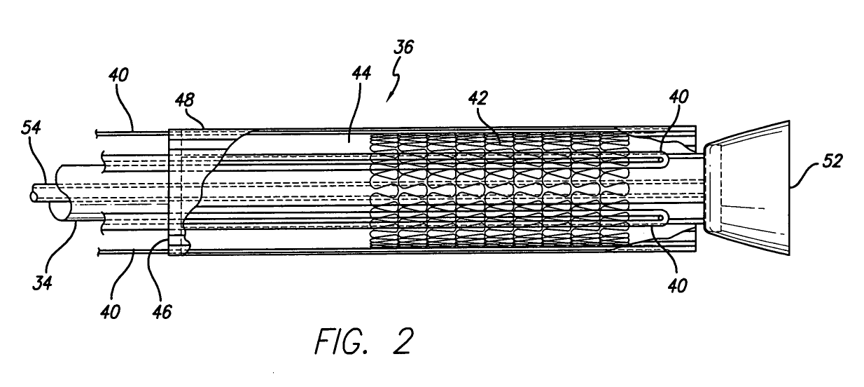

Preferably, the plurality of push rods 40 extend in a distal direction from the control assembly 38 and pass through a housing 36. With reference also to FIG. 2, a cardiac harness 42 is releasably supported on the distal end portions of the elongate push rods 40 in a compacted configuration within the housing 36. Preferably, the cardiac harness 42 comprises an elastic sleeve configured to fit around the heart and to exert a compressive force on the heart. In the illustrated embodiment, the harness 42 comprises several interconnected rows of undulating elastic members. Preferred cardiac harnesses are described in greater detail in U.S. patent application Ser. No. 09/634,043, filed Aug. 8, 2000 now U.S. Pat. No. 6,702,732; U.S. application Ser. No. 10/242,016, filed Sep. 10, 2002 now U.S. Pat. No. 6,723,041; U.S. application Ser. No. 10/287,723, filed Oct. 31, 2002; and U.S. application Ser. No. 10/656,722, filed Sep. 5, 2003, the entirety of each of which are incorporated by reference herein. It is to be understood that aspects of the delivery device 30 discussed herein can be used in connection with several other types of cardiac harnesses.

The term “cardiac harness” as used herein is a broad term that refers to a device fit onto a patient's heart to apply a compressive force on the heart during at least a portion of the cardiac cycle.

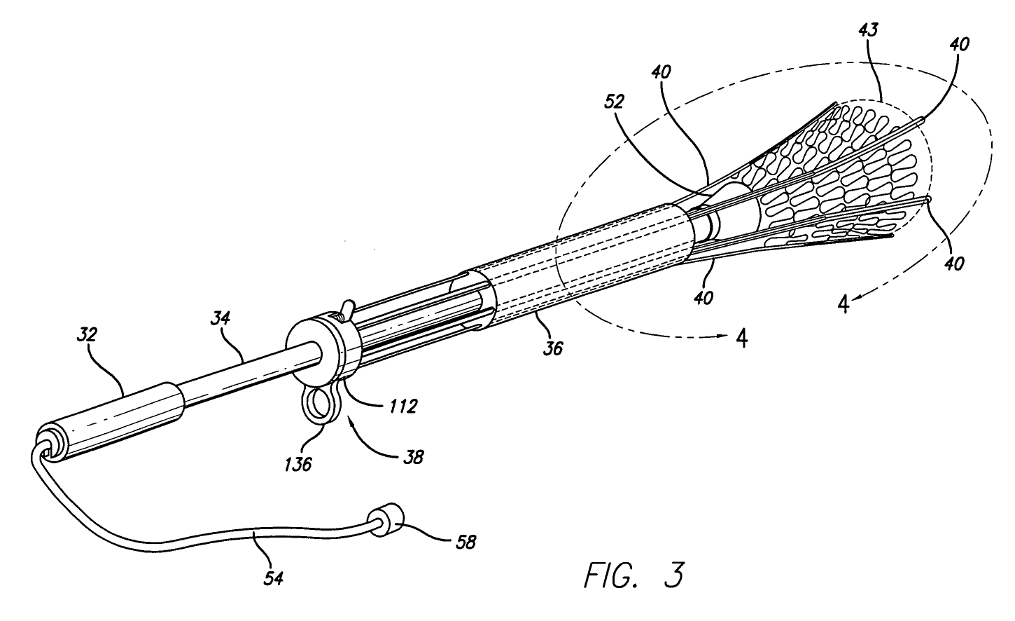

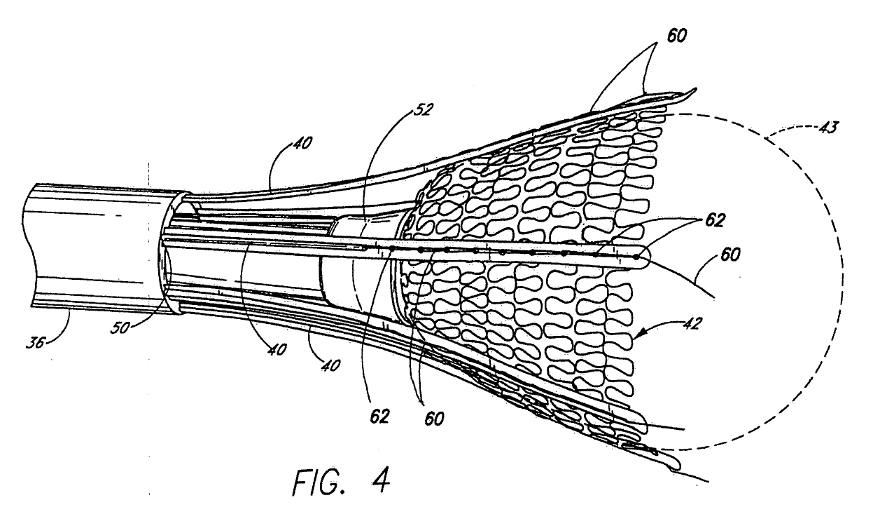

The control assembly 38 and plurality of push rods 40 are movable axially with respect to the shaft 34 from the retracted position, as illustrated in FIGS. 1 and 2, to an advanced, or deployed position, as illustrated in FIGS. 3 and 4. Thus, the delivery device 30 is configured to deploy the cardiac harness 42 from a compacted configuration within the housing 36 to an expanded configuration outside of the housing 36 thereby delivering the cardiac harness 42 onto a heart 43 (illustrated schematically in FIGS. 3 and 4), as is described in greater detail below.

The handle 32 is fixed to the shaft 34 in the illustrated embodiment. However, it is to be understood that in other arrangements the handle 32 may be movable relative to the shaft 34 along with the control assembly 38. Additionally, another embodiment may not employ a handle 32. Further, with reference to FIG. 1, a stop 39 preferably is provided on the shaft 34. The stop 39 comprises a raised portion that engages the control assembly 38 so that the assembly 38 cannot move distally over the shaft 34 beyond the stop 39. As such, the harness 42 is not advanced too far over the heart 43.

With reference again to FIG. 2, the housing 36 preferably is a relatively thin-walled, tubular member. Desirably, the housing 36 is supported substantially concentric with the shaft 34 to define an interior cavity 44 between an inner surface of the housing 36 and an outer surface of the shaft 34. Preferably, the cavity 44 is sized and shaped to contain the cardiac harness 42 in a compacted configuration therein.

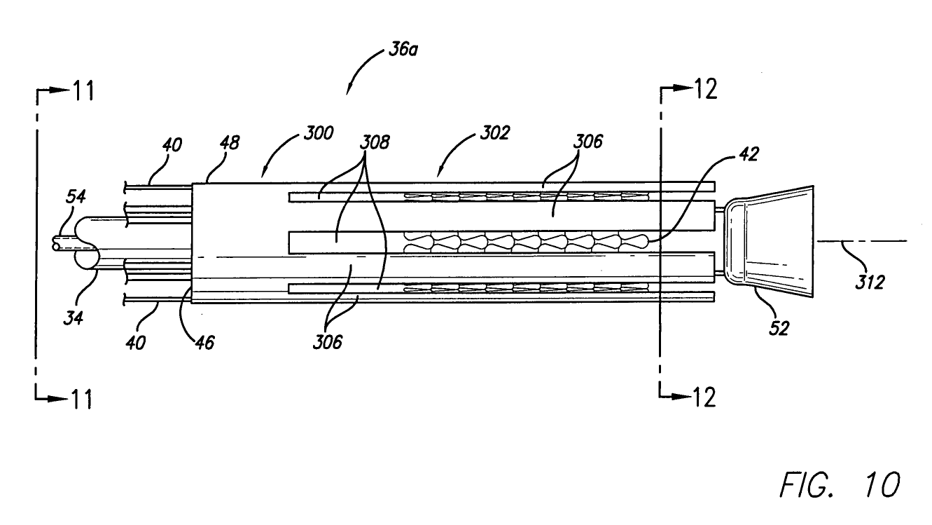

As indicated above, preferably the device 30 is configured to deliver the cardiac harness 42 in a minimally invasive procedure. Accordingly, a preferred housing 36 has a nominal outer diameter of less than about 5.1 cm (2 inches), more preferably, less than about 3.2 cm (1.25 inches). Preferably, the housing 36 is flexible such that its transverse cross-sectional shape may be collapsed or compressed as needed to advance through a minimally invasive surgical entry path, as described in greater detail below in relation to FIGS. 10-20. In the illustrated embodiments, the housing 36 is generally cylindrical in its relaxed or uncompressed condition. It is to be understood that, in another preferred embodiment, the housing is substantially elliptical in its relaxed condition such that the housing may have a cross-section with major axis and minor axis. This configuration may be especially beneficial for advancing the housing through body passages having relatively narrow clearance, such as advancing the housing between the ribs.

With continued reference to FIG. 2, a base portion 46 of the housing 36 preferably defines a closed end of the cavity 44 and supports the housing 36 relative to the shaft 34. The base end 46 may be secured to the shaft 34 by mechanical fasteners, adhesives or other suitable methods apparent to one of skill in the art. In one embodiment, the base end 46 is rotatable relative to the shaft 34. Preferably, the distal end of the housing is open to define an open, distal end of the cavity 44 to permit the cardiac harness 42 to be advanced from the cavity 44.

Preferably, an inner surface of the housing 36 defines a plurality of channels 50 (FIG. 4) extending axially throughout the length of the housing 36. Each of the channels 50 preferably is sized and shaped to slidably receive one of the plurality of push rods 40. Thus, preferably, the number of channels 50 is equal to the number of push rods 40. Further, each channel 50 preferably opens into a cavity 44 along at least a portion of the length of the channel 50.

In the embodiments illustrated, eight push rods 40 and eight channels 50 are provided and are substantially equally spaced around the circumference of the housing 36. A greater or lesser number of push rods 40 and channels 50 may be provided as appropriate to support and deploy a cardiac harness. In an additional arrangement, the channels 50 may be omitted and the push rods 40 may simply be restrained from moving radially outwardly by an outer wall 48 of the housing 36. Other suitable arrangements to guide the push rods 40 and house the cardiac harness 42 may also be used.

With continued reference to FIGS. 1-4, the delivery device 30 preferably includes a positioning arrangement configured to hold the delivery device 30 in a desired position relative to the heart 43. In the illustrated arrangement, the positioning arrangement comprises a suction cup member 52 supported on a distal end of the shaft 34. A tube 54 extends through the shaft 34 and is connected to the suction cup member 52. A distal end of the tube 54 opens into an interior space defined by the suction cup member 52. The proximal end of the tube 54 includes a connector 58 that allows connection of the tube 54 to a pump member such as a syringe or other source of vacuum. Accordingly, once the delivery device is properly positioned, air may be withdrawn from within the tube 54 to create a vacuum condition within the interior space of the suction cup member 52, thereby permitting the suction cup member 52 to securely hold the heart of a patient.

A clip 56 secures the tube 54 relative to the handle 32 to prevent the proximal end of the tube 54 from passing through the shaft 34. Thus, the clip 56 also operates to secure the suction cup member 52 to the delivery device 30. In a preferred embodiment, the tube 54 and suction cup member 52 are not rigidly affixed to the shaft 34 so that the shaft 34 may be moved relative to the tube 54 and suction cup 52. In another embodiment, the shaft 34 and a proximal end of the suction cup 52 are threaded so that the suction cup may be threaded onto the shaft. In still other embodiments, other structure may be used to releasably connect the suction cup to the shaft.

Preferably, the cardiac harness 42 is secured to a distal portion of each of the plurality of push rods 40 by a flexible line that is configured into a releasable stitch, such as described in U.S. application Ser. No. 10/715,150, filed Nov. 17, 2003, the entirety of which is incorporated by reference herein. Desirably, as shown in FIG. 4, the flexible line 60 passes through a plurality of openings 62 in the distal portion of the push rod 40 and is arranged into a series of interconnected loops that are releasable by actuation of the control assembly 38 in a manner described in greater detail below. Release of the interconnected loops, in turn, releases the cardiac harness 42 from the push rods 40.

With particular reference to FIGS. 1 and 3, the control assembly 38 preferably includes a substantially cylindrical body portion 112 and a release member 136. A portion of the release member 136 preferably is received within a cavity of the body portion 112. An exposed pull portion of the release member 136 extending outwardly from the body portion 112 is generally annular in shape, such that a user of the delivery device 30 can grasp the release member 136 with one or more fingers extending through the a hole defined by the annular shape. As the release member 136 is pulled away from the body portion 112 of the control assembly 38, the release member 136 pulls on the flexible lines 60 such that the interconnected loops of the releasable stitch are unraveled.

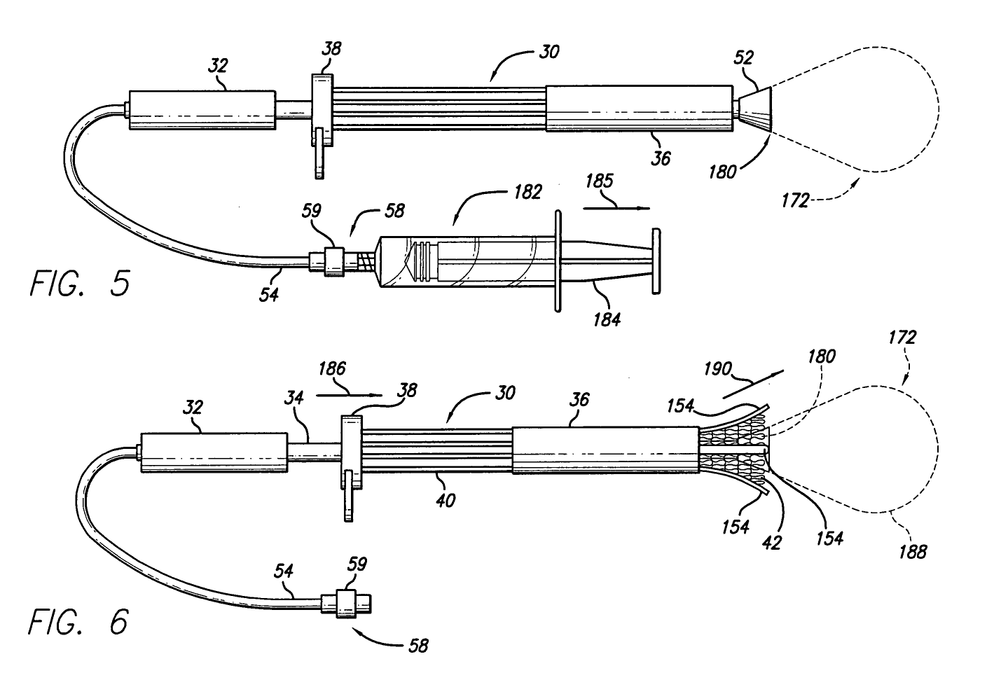

FIGS. 5-9 illustrate the use of a delivery device 30, preferably configured substantially as described above, to deliver a cardiac harness 42 onto a heart 172. Preferably, the delivery device 30 is configured to locate and grasp the heart 172, accurately position the cardiac harness 42 onto the heart 172, and permit withdrawal of the delivery device 30 without disturbing the positioning of the cardiac harness 42.

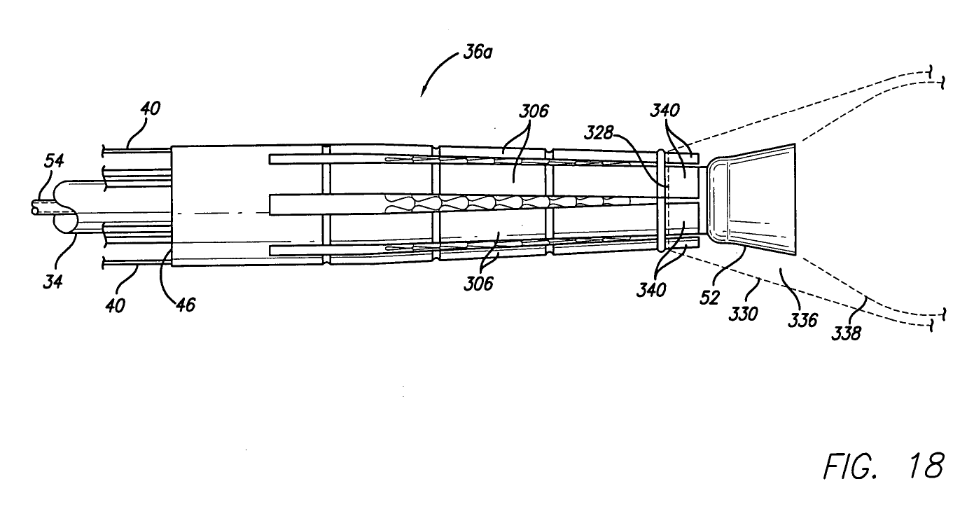

With reference to FIG. 5, preferably, the suction cup 52 of the delivery device 30 engages an apex portion 180 of the heart 172, which is illustrated schematically in FIGS. 5-9. The distal end of the delivery device 30 may access the heart 172 through any suitable method, but preferably through a minimally invasive procedure such as that described in relation to FIGS. 17, 18 and 20. In FIGS. 5-9, the pericardial sac or pericardium 174 surrounding the heart is omitted for ease of illustration.

A pump device, such as a syringe 182, is connected to the tube 54 through the connector 58. Desirably, the syringe 182 is connected to the tube 54 with the plunger 184 in a compressed position. Once connected, the plunger 184 is retracted (as indicated by the arrow 185 in FIG. 5) to create a vacuum condition within the tube 54 and, thus, within the space defined by the interior of the suction cup member 52. Due to the vacuum condition, the suction cup member 52 grasps the apex 180 such that the heart 172 is held in a desired position relative to the delivery device 30.

Preferably, the connector 58 includes a one-way valve 59 that is configured to inhibit air from flowing from the syringe to the tube 54 through the connector 58. Accordingly, the syringe 182 may be removed from the tube 54 once a vacuum condition has been created. Although a syringe 182 is preferred as a pump member due to its simplicity and low cost, other suitable pump devices may also be used to create a vacuum within the tube 54, as will be appreciated by one of skill in the art.

With reference next to FIG. 6, once the delivery device 30 has been properly secured to the apex portion 180 of the heart 172, the control assembly 38 may be advanced, relative to the shaft 34, toward the heart 172, as indicated by the arrow 186 in FIG. 6. The plurality of push rods 40 are advanced toward the heart 172 with the control assembly 38 thereby advancing the cardiac harness 42 from its compacted configuration within the housing 36 onto the heart 172 in a direction from the apex portion 180 to the base portion 188, as indicated by the arrow 190 in FIG. 6. As shown, the harness 42 preferably stretches elastically to fit over the heart. However, it is to be understood that a substantially non-elastic harness embodiment can also be delivered by this device.

As illustrated in FIG. 6, the plurality of push rods 40 splay outwardly to conform to the shape of the heart 172 as they are advanced relative to the shaft 34 of the delivery device 30. Preferably the tips 154 of the push rods 40 are canted at an outward angle θ relative to the remainder of the push rod 40 such that contact of the tip 154 with the heart 172 is generally avoided, thereby preventing trauma to the heart 172.

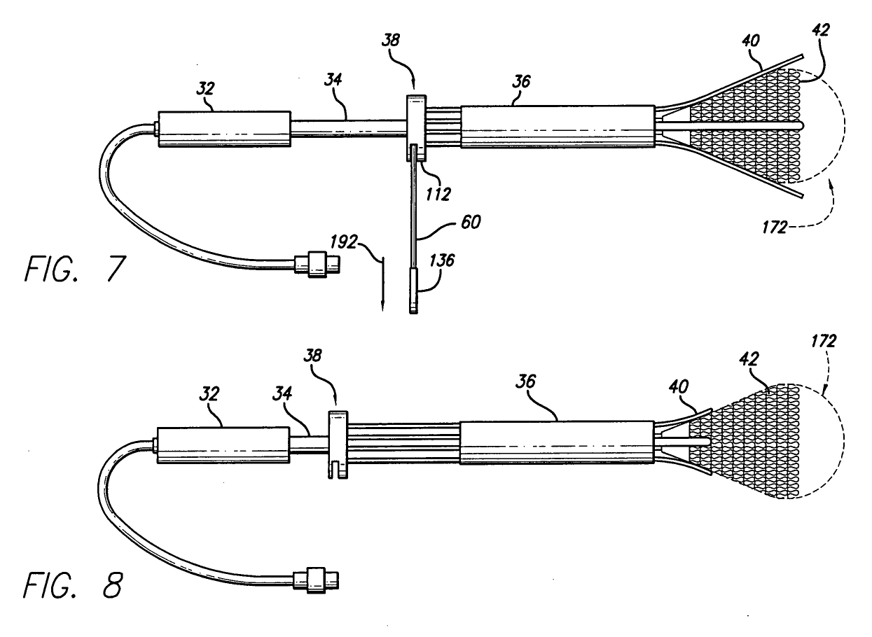

With reference to FIG. 7, the control assembly 38 continues to be advanced until the cardiac harness 42 is properly positioned on the heart 172. Once the cardiac harness 42 is properly positioned, the release member 136 is pulled away from the body portion 112 of the control assembly 38, as indicated by the arrow 192. Accordingly, the cardiac harness 42 is released from the plurality of push rods 40.

With reference to FIG. 8, once the cardiac harness 42 has been released from the plurality of push rods 40, the generally-elastic harness preferably contracts onto the heart. The control assembly 38 is then retracted relative to the shaft 34 to retract the plurality of push rods 40 from the cardiac harness 42, which remains on the heart 172. As noted above, preferably, the push rods 40 are configured such that retraction of the push rods 40 does not tend to pull the cardiac harness 42 from its desired position on the heart 172. Specifically, in the illustrated embodiment, the outwardly canted tips 154 of the plurality of push rods 40 help prevent the push rods 40 from exerting a pulling force on the cardiac harness 42.

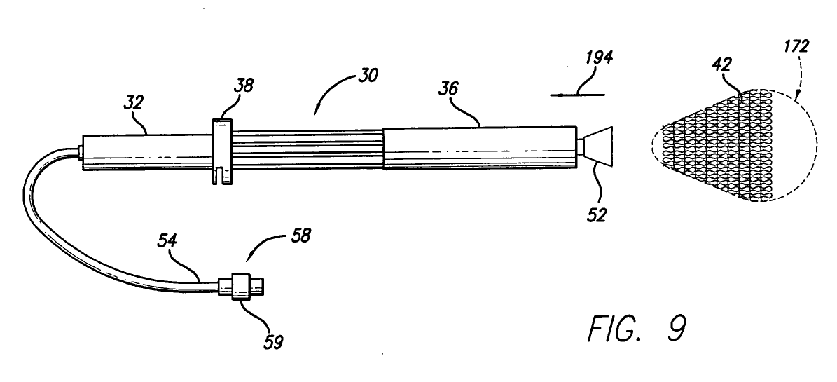

With reference to FIG. 9, once the plurality of push rods have been fully retracted from the cardiac harness 42 and the heart 172, the one-way valve 59 within the connector 58 may be opened to release the vacuum condition within the tube 54 and suction cup member 52. As a result, the delivery device 30 may be removed from the heart 172, as indicated by the arrow 194 in FIG. 9, as the suction cup member 52 is no longer grasping the heart 172. Thus, the delivery device 30 is retracted from the heart, leaving the cardiac harness 42 in place.

As discussed above, the housing 36 has a collapsible cross-sectional shape. To facilitate insertion of the delivery device 30 through a minimally invasive surgical entry path, the distal end of the housing may be compressed or collapsed to a circular cross-section with a diameter that is smaller than the diameter of the base end 46 at the proximal end of the housing 36. To facilitate advancement through a narrow passage in a minimally invasive surgical entry path, such as between two ribs of a patient, the housing may be flattened to an oval or substantially elliptical cross-section with a minor axis and major axis. As the housing 36 is advanced past a narrow passage, its distal end returns to a circular cross-sectional shape and portions of the housing adjacent to the narrow passage flatten to allow further advancement of the housing. It will be appreciated that, compared to a rigid housing, a housing with a collapsible cross-section shape places less stress on tissues and bones along the minimally invasive surgical path and, thus, is likely to result in lower incidence of injury or trauma.

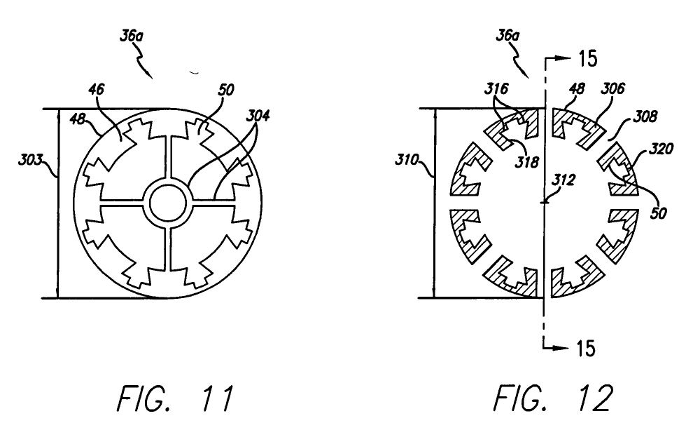

In one embodiment, as shown in FIG. 10, a housing 36a is substantially tubular and has a length sufficient to contain an entire cardiac harness 42 in a compacted configuration while the cardiac harness is attached to the push rods 40. Preferably, the housing 36a has a rigid portion 300 adjacent to the proximal end of the housing and a flexible portion 302 adjacent to the distal end of the housing. There is shown in FIG. 11 a plan view of the proximal end of the housing 36a taken in the direction of line 11-11 of FIG. 10. The base end 46 at the rigid portion 300 of the housing 36a is substantially circular in cross-sectional shape with an outer diameter 303 sufficient to accommodate a circular arrangement of the push rods holding a cardiac harness in a compacted configuration. In one embodiment, the outer diameter 303 of the rigid portion 300 is about 3.18 cm (1.25 in). The push rods slide in a plurality of channels 50 extending axially along the length of the housing 36a. A support member 304 fixedly attached to the rigid portion 300 supports the housing 36a in a substantially concentric position relative to the shaft 34 (not shown).

Referring again to FIG. 10, the flexible portion 302 of the housing 36a includes a plurality of elongate slats 306 separated from each other by cut-out portions or gaps 308. In the illustrated embodiment, the flexible portion is of a length that preferably is at least 50% of the length of the housing 36a, and more preferably at least about 70% of the length of the housing 36a. Preferably, the flexible portion is of a length sufficient to traverse a minimally invasive surgical entry path to the heart. There is shown in FIG. 12 a transverse cross-sectional view of the flexible portion 302 taken in a direction toward the distal end of the housing 36a. The outer wall 48 along the flexible portion 302 defines a substantially circular cross-sectional arrangement or shape with a nominal or relaxed diameter 310 that is, preferably, equivalent to the diameter 303 of the rigid portion 300. The gaps 308 are sized to allow the slats 306 to bend radially inward toward a longitudinal, central axis 312 of the housing 36a. The gaps 308 allow the relaxed diameter 310 of the flexible portion 302 to increase or decrease to a non-relaxed diameter 310′ when radial forces are applied to the slats 306.

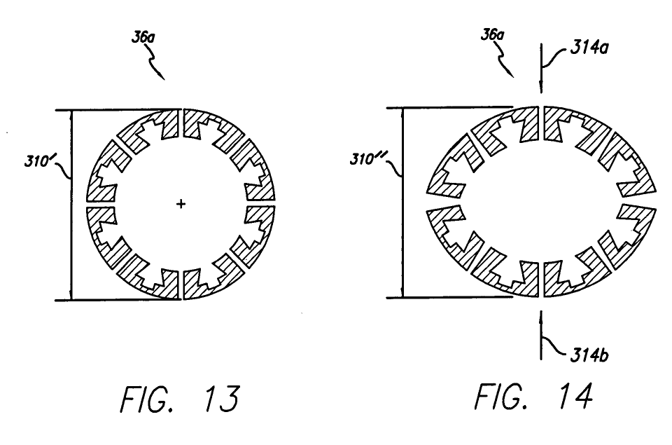

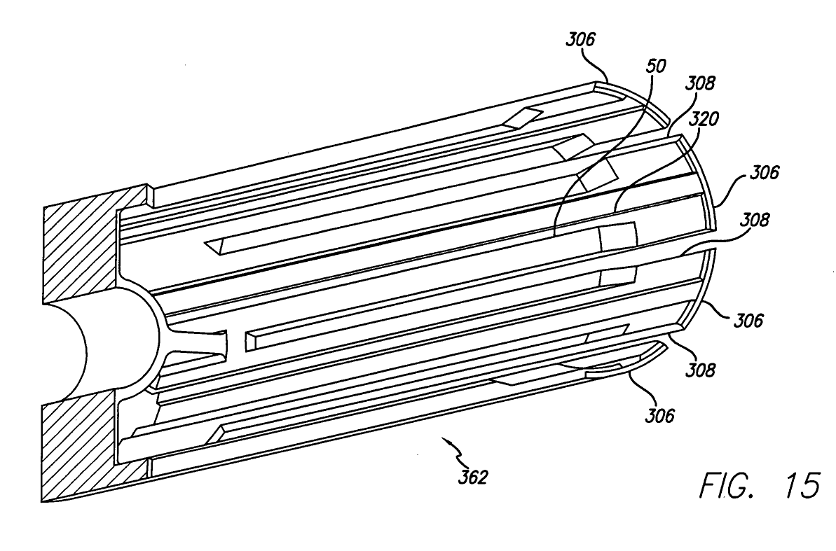

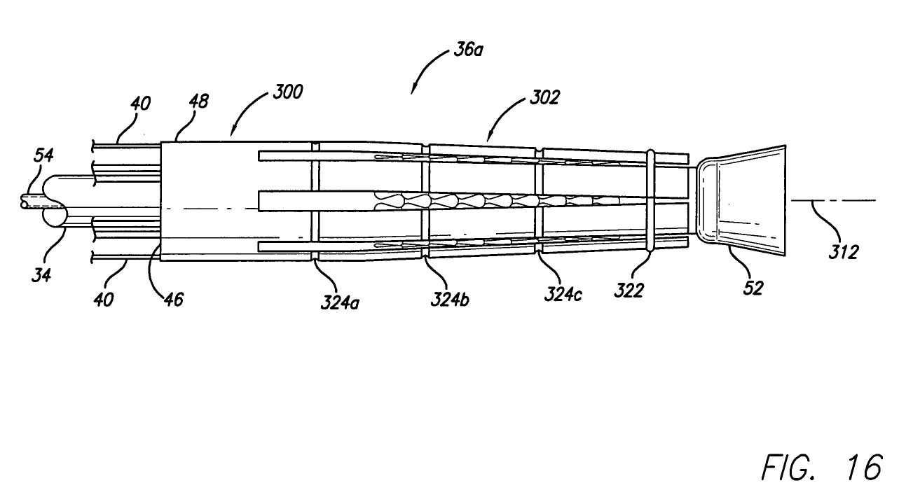

As shown in FIG. 13, a user of the delivery device 30 may compress or collapse the distal end or other section of the flexible portion 302 of the housing 36a to a non-relaxed diameter 310′ that is smaller than the relaxed diameter 310. Such compression facilitates inserting the delivery device 30 into an incision. As shown in FIG. 14, the outer wall 48 may define a substantially elliptical cross-section along a section of the flexible portion 302 where radial or transverse forces are applied to the slats 306 in opposing directions, as shown by arrows 314a and 314b. Preferably, a minor axis 310″ of such a substantially elliptical cross-section is less than the relaxed diameter 310. Both the non-relaxed diameter 310′ in FIG. 13 and the minor axis 310″ in FIG. 14 can be referred to as a minimum cross-sectional dimension, which is defined herein as the shortest possible linear distance between any two points on the perimeter of the cross-sectional shape and passing through the center of the cross-sectional shape. It is to be understood that other cross-sectional shapes may be achieved by compressing a collapsible housing. With any cross-sectional shape, when it is desired to advance the housing 36a between two ribs or other narrow passage of a minimally invasive surgical entry path, the minimum cross-sectional dimension is preferably less than a distance across the narrow passage.

Referring again to FIG. 12, each of the slats 306 comprises one channel 50 for holding a push rod 40. One gap 308 is positioned between two channels 50. As such, the number of slats 306 and gaps 308 is equivalent to the number of channels 50 and push rods 40. However, the numbers need not be equivalent. It will be appreciated by persons skilled in the art that the ease and extent to which the flexible portion 302 is compressed depends at least in part on the width and the number of the gaps 308. For example, eight gaps each having a width of 0.33 cm (0.13 in) will allow for a reduction of 1.26 cm (0.50 in) from the relaxed diameter when the flexible portion 302 is compressed. If the relaxed diameter is 3.18 cm (1.25 in) in this example, the distal end of the housing 36a can be compressed to a non-relaxed diameter of 1.92 cm (0.75 in). While eight gaps 308 are illustrated, it is to be understood that a lesser or greater number may be employed with smaller or larger widths to achieve a desired degree of flexibility and a desired reduction from the relaxed diameter. Thus, it is contemplated that four to twelve gaps may be employed.

With continued reference to FIG. 12, the channels 50 have a bottom surface 316 and a top opening 318. The opening 318 is narrower than the bottom surface 316 such that the channel 50 has a dovetail cross-sectional shape that holds a correspondingly shaped push rod 40 (not shown) in the channel 50 while the push rod slides therein. In one embodiment, the flexible lines 60 attaching the cardiac harness 42 to the push rods 40 creates a series of releasable loops along the outward facing surface of the push rod adjacent to the bottom surface 316. A narrow groove 320 running along the center of the bottom surface 316 provides clearance for the loops as the push rod 40 slides through the channel 50.

As noted above, the tips 154 of the push rods 40 are canted outward or radially away from the center axis 312 of the housing 36a. As shown in FIG. 15, the channels 50 terminate at a distance away from the distal end of the housing 36a. Preferably, the distance is sufficient to allow the canted tips 154 of the push rods to be contained within the housing 36a. As the push rods 40 move longitudinally out of the housing 36a, curved portions adjacent to the canted tips 154 slide against the heart and facilitate bending of the push rods 40 such that push rods 40 splay radially away from the center axis 312 and conform to the curvature of the heart.

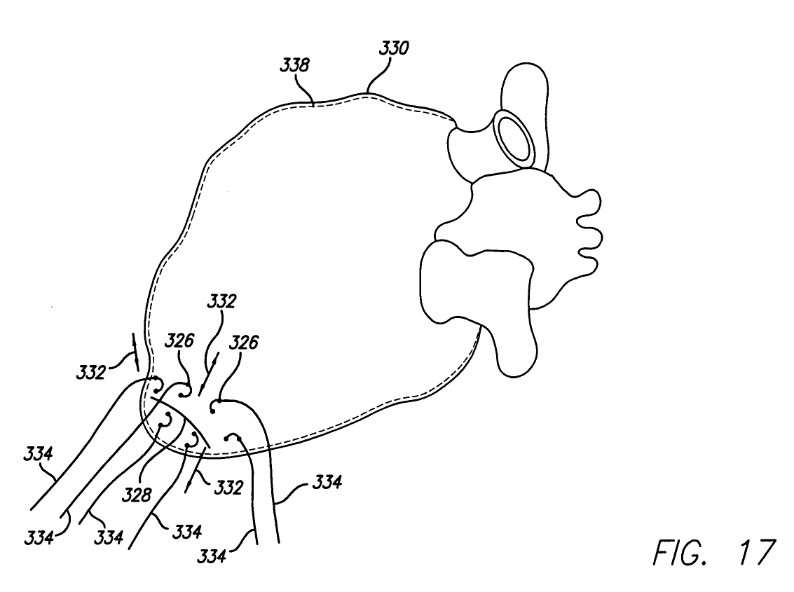

Referring to FIG. 16, in a further embodiment an annular biasing member 322, such as an elastic ring, is disposed adjacent to the distal end of the housing 36a. The biasing member 322 applies a compressive or radially inward force to the slats 308 such that the flexible portion 302 tapers down toward the distal end of the housing 36a. The outer wall 48 beneath the biasing member may have a notch (not shown) extending circumferentially across the slats. The notch is configured to keep the biasing member 322 in a desired position on the flexible portion 302 of the housing 36a. There may be additional notches 324a, 324b, 324c at various locations along the flexible portion 302 to facilitate changing the position of the biasing member 322 or to facilitate the installation of one or more additional biasing members to customize the profile of the housing 36a as appropriate for a minimally invasive surgical entry path.

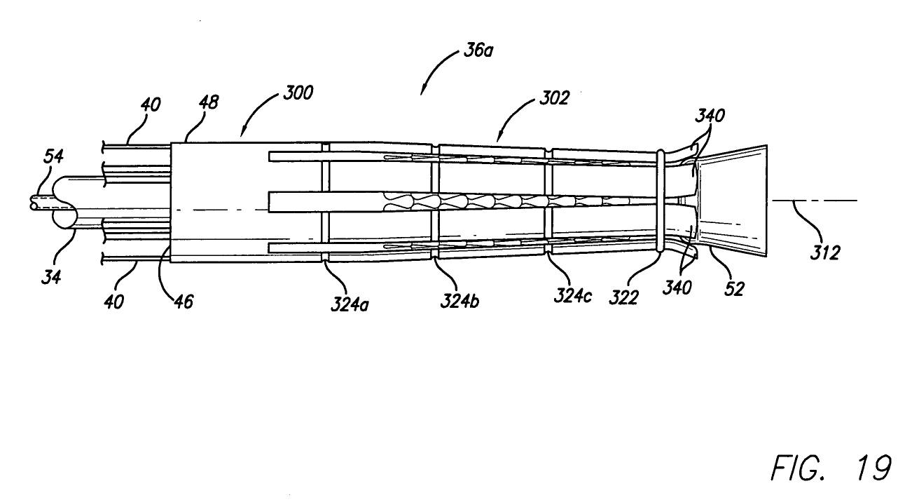

After advancing through a minimally invasive surgical entry path, the suction cup member 52 is advanced through an incision in the pericardium of the heart. As shown in FIG. 17, a plurality sutures 326 may be used to reinforce and hold open an incision 328 in the pericardium 330 of the heart to allow the suction cup member 52 and the distal ends of the slats 306 to advance through the incision. The sutures 326 are placed around the incision 328. Preferably, holes formed in the pericardium when making the sutures are in a line substantially perpendicular to the incision as shown by arrows 332, but the suture holes can have other orientations as well. It is also preferred that the number of sutures 326 corresponds to the number of slats 306. Each suture 326 has a pull line 334 extending from the suture and through the minimally invasive surgical entry path to a position outside the patient. The sutures 326 and pull lines 334 are formed of any suitable biocompatible and/or bioabsorbable material well known in the art. One or more of the pull lines 334 can be manipulated by the doctor to help open the incision to receive the suction cup member 52 and the distal ends of the slats 306. In addition, as shown in FIG. 18, the sutures 326 may be pulled proximally to tent the pericardium, that is, to create a space 336 between a portion of the pericardium 330 and a portion of the epicardial surface 338 of the heart. For ease of illustration, the sutures 326 are not shown and the pericardium 330 and epicardial surface 338 are shown schematically as dashed lines in FIG. 18. The space 336 allows the push rods 40 to advance an attached cardiac harness 42 around a portion of the epicardial surface 338 of the heart. The sutures 326 may also be manipulated by the doctor to tighten the incision 328 around the distal end of the slats 306. Tightening the incision ensures that as the push rods 40 slide out of the housing 36a as they advance the cardiac harness 42 into the space 336.

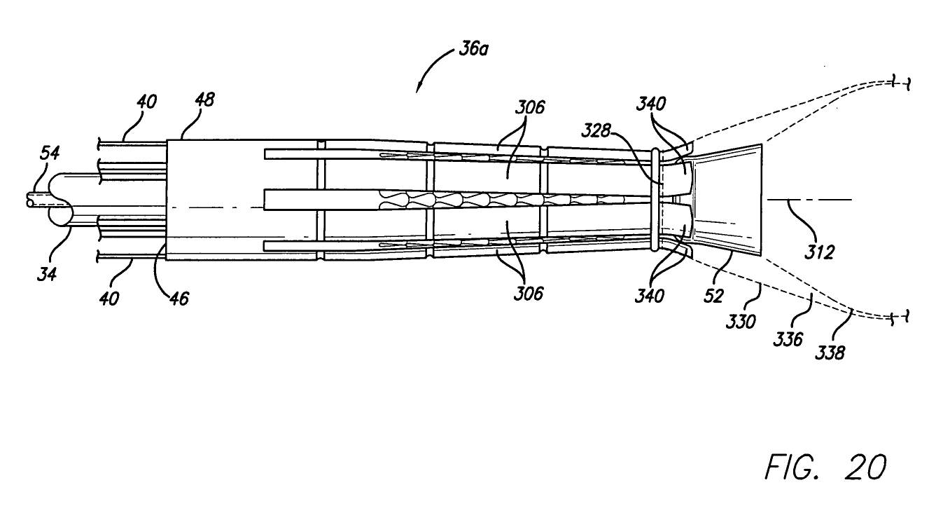

Referring to FIG. 19, in another embodiment the slats 306 have curved end portions 340 adjacent to the distal end of a housing 36b. The curved end portions 340 are curved outward or radially away from the center axis 312 of the housing 36b. Preferably, the outer wall 48 at the curved end portions 340 defines a substantially circular cross-section with a diameter no greater than the diameter of the suction cup member 52 located distally from the curved end portions 340. In practice, the user of the apparatus may compress the curved end portions 340 to an elliptical cross-sectional shape in order to insert one side of the distal end of the housing 36b into the incision 328 first, followed by the remaining side. As shown in FIG. 20, the curved portions 340 are configured to lock beneath the pericardium 330 and to pull the interior surface of the pericardium 330 adjacent to the incision 328 so as to create a space 336 between the pericardium 330 and a portion of the epicardial surface 334 of the heart. For ease of illustration, the pericardium 330 and epicardial surface 338 are shown schematically as dashed lines in FIG. 20. Preferably, the corners along the edges of the curved end portions 340 are rounded to facilitate insertion through the incision 328 and to avoid puncturing the pericardium 330. With the curve end portions 340 locked beneath the pericardium 330, the push rods 40 may extend the cardiac harness 42 into the space 336. As the push rods 40 make contact with the heart and splay outwardly, the push rods 40 exert an outward radial force on the curved end portions 340 in opposition to the inward radial force exerted by the biasing member 332. As a result, the curved end portions 340 preferably splay outwardly, but only slightly. When the curved end portions 340 splay slightly, the push rods 40 are allowed to conform more readily over a portion of the epicardial surface of the heart. As a further result, the curved end portions 340 are pressed further against the interior surface of the pericardium 330 adjacent to the incision 328, which serves to better secure the curved portions 340 beneath the pericardium 330.

While the illustrated embodiments shown in FIGS. 10-20 have a housing with flexible slats separated by gaps, it will be appreciated by persons of skill in the art that other housing structures may be used resulting in a collapsible cross-sectional shape. For example, it is contemplated that a housing may comprise a thin-walled sleeve configured to fold or stretch along a length of the sleeve.

Although the delivery device 30 is especially well suited for use in a minimally invasive delivery procedure, the device 30 may also be used for open chest procedures, wherein the sternum of the patient is split to provide access to the heart 172. In addition, although the device 30 described herein utilizes a plurality of push rods 40, other suitable structures may also be used to support the cardiac harness 42 when being advanced over the heart. For example, an expandable sleeve can serve as a support structure. Furthermore, it is to be understood that a cardiac harness 42 may be releasably supported in an expanded, or substantially expanded, configuration to a variety of support structures by the releasable stitch referred to herein, or by a similar releasable stitch arrangement.

In the embodiments disclosed herein, the illustrated cardiac harness 42 is formed of several rows of elastic elements. The illustrated harness comprises undulating wire arranged in several adjacent rings, each of which comprises an elastic row. As illustrated, the harness 42 is releasably attached to the push rods by a stitch being wound around some or all of the rows. Of course, it is to be understood that aspects of the present invention can be employed with harnesses having different structure than the illustrated harness, which is included for example only. For example, any harness having one or more openings that could accommodate the releasable stitch could be used such as, for example, a harness formed of a woven or non-woven fibrous material and/or a harness formed of a mesh, honeycomb or other type of material.

Although the present invention has been described in the context of a preferred embodiment, it is not intended to limit the invention to the embodiment described. Accordingly, modifications may be made to the disclosed embodiment without departing from the spirit and scope of the invention. It is also contemplated that various combinations or subcombinations of the specific features and aspects of the embodiments can be combined with or substituted for one another in order to form varying modes of the invention. Accordingly, the invention is intended to be defined only by the claims that follow.

What is claimed:

1. An apparatus for delivering a cardiac harness onto a heart, comprising:
an elongate body having a proximal portion and a distal portion, the distal portion having a tubular housing sized to contain the cardiac harness in a compacted configuration, the housing having a proximal end, an open distal end, an inner surface, and an outer surface, the outer surface defining a collapsible cross-sectional shape; and
a plurality of elongate push rods longitudinally movable with respect to the elongate body, the cardiac harness releasably connected to each of the push rods such that advancement of the push rods in a distal direction moves the cardiac harness from the compacted configuration in the housing to an expanded configuration outside the housing.

2. The apparatus of claim 1, wherein the cross-sectional shape is of a size suitable for a minimally invasive procedure.

3. The apparatus of claim 1, wherein the housing is substantially cylindrical with a diameter less than 5.1 cm (2 inches).

4. The apparatus of claim 3, wherein the diameter of the housing is less than 3.2 cm (1.25 inches).

5. The apparatus of claim 1, wherein the inner surface defines a plurality of channels sized and shaped to receive the push rods.

6. The apparatus of claim 5, wherein each of the channels has a surface that defines a cross-section having a first shape and each of the push rods has a surface that defines a cross-section having a second shape that is substantially the same as the first shape.

7. The apparatus of claim 6, wherein at least one of the first shape and the second shape comprises a dovetail form.

8. The apparatus of claim 1, wherein the housing comprises a plurality of elongate slats extending longitudinally to the distal end of the housing, the slats spaced apart and forming a perimeter around the housing.

9. The apparatus of claim 8, wherein the slats are biased to form a smaller perimeter at the distal end of the housing relative to the proximal end of the housing.

10. The apparatus of claim 8, wherein the slats comprise curved end portions at the distal end of the housing, the curved end portions configured to temporarily lock the distal end of the housing in an incision in a pericardial sac surrounding the heart.

11. The apparatus of claim 8, wherein the slats have a longitudinal length greater than 50% of a longitudinal distance between the proximal end and the distal end of the housing.

12. The apparatus of claim 11, wherein the longitudinal length of the slats is greater than 70% of the longitudinal distance between the proximal end and the distal end of the housing.

13. The apparatus of claim 1, wherein the housing has a substantially circular cross-sectional shape having a diameter, and wherein at least a portion of the housing is compressible to a substantially elliptical cross-sectional shape having a minor axis that is less than the diameter.

14. The apparatus of claim 1, wherein the housing has a cross-sectional shape having a first perimeter, and wherein at least a portion of the housing is compressible to a reduced cross-sectional shape having a second perimeter that is less than the first perimeter.

15. The apparatus of claim 1, further comprising an annular biasing member coupled to the outer surface of the housing, the biasing member compressing a portion of the housing to a smaller cross-sectional shape relative to an uncompressed portion of the housing.

16. An apparatus for delivering a cardiac harness onto a heart, comprising:
a tubular housing sized to contain the cardiac harness in a compacted configuration, the housing having a proximal end, an open distal end, an inner surface, and an outer surface, the outer surface defining a collapsible cross-sectional shape; and
an elongate support member longitudinally movable with respect to the housing, the cardiac harness releasably connected to the support member such that longitudinal movement of the support member in a distal direction moves the cardiac harness from the compacted configuration in the housing to an expanded configuration outside the housing.

17. The apparatus of claim 16, wherein the cross-sectional shape is adapted for advancing through a minimally invasive surgical entry path.

18. The apparatus of claim 16, wherein the cross-sectional shape is substantially circular with a diameter less than 5.1 cm (2 inches).

19. The apparatus of claim 18, wherein the diameter of the cross-sectional shape is less than 3.2 cm (1.25 inches).

20. The apparatus of claim 16, wherein the support member comprises a plurality of flexible push rods having a sliding, interlocking relationship with the inner surface of the housing.

21. The apparatus of claim 20, wherein the push rods have a cross-section having a first shape and the inner surface of the housing defines a plurality of channels each with a cross-section having a second shape substantially similar to the first shape.

22. The apparatus of claim 21, wherein at least one of the first shape and the second shape comprises a dovetail form.

23. The apparatus of claim 16, wherein the housing comprises a plurality of longitudinal slats spaced part in a circumferential arrangement around the housing.

24. The apparatus of claim 23, wherein the slats are biased in a smaller circumferential arrangement at the distal end of the housing relative to the proximal end of the housing.

25. The apparatus of claim 23, wherein the slats comprise curved end portions at the distal end of the housing, the curved end portions sized to advance through an incision in a pericardial sac surrounding the heart and configured to push in a distal direction against a portion of the pericardial sac adjacent to the incision.

26. The apparatus of claim 23, wherein the slats have a longitudinal length greater than 50% of a longitudinal distance between the proximal end and the distal end of the housing.

27. The apparatus of claim 26, wherein the longitudinal length of the slats is greater than 70% of the longitudinal distance between the proximal end and the distal end of the housing.

28. The apparatus of claim 16, wherein the housing has a cross-sectional shape having a first dimension, the first dimension equivalent to the shortest possible linear distance between any two points on the perimeter of the cross-sectional shape and passing through the center of the cross-sectional shape, and wherein at least a portion of the housing is collapsible to a reduced cross-sectional shape having a second dimension that is less than the first dimension, the second dimension equivalent to the shortest possible linear distance between any two points on the perimeter of the reduced cross-sectional shape and passing through the center of the reduced cross-sectional shape.

29. The apparatus of claim 16, wherein the housing tapers from a first cross-sectional shape having a first perimeter at the proximal end of the housing to a second cross sectional shape having a second perimeter at the distal end of the housing, the second perimeter being smaller in size than the first perimeter.

30. An apparatus for delivering a cardiac harness onto a heart, comprising:
a housing having an outer wall defining a proximal end and a distal end, the outer wall having a flexible portion adjacent the distal end, the flexible portion defining a reduced orientation having a first diameter, the first diameter being of a size sufficient to permit the apparatus to pass through a minimally invasive surgical entry path;
a support member sized and shaped to be contained in the housing, the cardiac harness releasably connected to the support member such that longitudinal movement of the support member in a distal direction (a) moves the cardiac harness from the compacted configuration in the housing to an expanded configuration outside the housing, and (b) urges the flexible portion into an expanded orientation having a second diameter that is larger than the first diameter.
